(19) United States
(12) Patent Application Publication
(10) Pub. No.: US 2004/0220249 A1
(43) Pub. Date: Nov. 4, 2004

(54) Prevention of surgical adhesions using selective COX-2 inhibitors

(75) Inventors: Mark Puder, Medfield, MA (US); Arin K. Greene, Wellesley, MA (US); Judah Folkman, Brookline, MA (US)

Correspondence Address: David S. Resnick, NIXON PEABODY LLP, 100 Summer Street, Boston, MA, 02110, US

(73) Assignee: Children's Medical Center Corporation, Boston, MA (US)

(21) Appl. No.: 10/767,870

(22) Filed: Jan. 29, 2004

Related U.S. Application Data
(60) Provisional application No. 60/443,345, filed on Jan. 29, 2003.
(60) Provisional application No. 60/512,379, filed on Oct. 17, 2003.

(51) Int. Cl. A61K 31/415

(52) U.S. Cl. 514/406; 424/422; 514/403

(57) ABSTRACT

The present invention provides a method for the minimization or prevention of adhesion formation during or following a surgical procedure. The method comprises administering to the patient in need thereof a therapeutically effective amount of at least one COX-2 inhibitor, wherein the COX-2 inhibitor is not nimesulide. The COX-2 inhibitor can be administered before, during, or after surgery. In a preferred embodiment, the COX-2 inhibitor is celecoxib, also known as Celebrex (Pfizer).

### CROSS REFERENCE PARAGRAPH

This application claims the benefit of U.S. Provisional Application No. 60/443,345, filed Jan. 29, 2003 and U.S. Provisional Application No. 60/512,379.

### BACKGROUND OF THE INVENTION

Adhesion formation, the joining of two normally separate surfaces due to trauma or inflammation, is a major problem following surgical procedures. Adhesions following surgery frequently cause postoperative pain, blockage of intestines, and infertility. Adhesions are the major cause of intestinal obstruction and it is estimated that following an intra-abdominal procedure, adhesions occur in some 50 to 80 percent of patients. Intestinal obstruction caused by adhesions leads to prolonged hospital stays, additional abdominal surgery, and even death. Abnormal scarring in the abdomen also increases the morbidity of future surgery because adhesions lead to increased blood loss and injury to internal organs. Adhesion formation is also problematic in orthopedic and plastic surgeries, such as in the hand, where impediment of movement is frequently troublesome to the patient.

Intra-abdominal adhesions are the leading cause of secondary infertility and responsible for up to 20% of infertility cases (Ray 1998, Ellis 1999). Abnormal scarring in the abdomen increases the morbidity of future surgery because adhesiolysis may lead to increased blood loss and injury to internal organs. Adhesion formation may also cause additional morbidity in extra-abdominal procedures, such as in the hand, where impediment of movement is frequently troublesome to the patient. The prevention of adhesions would profoundly decrease morbidity and reduce health care costs across a broad range of medical disciplines (Menzies et al 2001).

After injury to the peritoneal lining, the entire epithelial lining becomes re-epithelialized with mesothelial cells and is complete in 5-6 days. Peritonal injury may result in local ischemia, deposition of polymorphonuclear leukocytes, macrophages, fibrin, mesenchymal cells, fibroblasts and new blood vessels resulting in adhesion formation. Eventually the adhesion matures into a mesothelial covered fibrous band, (dizerega, G S and Campeau, J D) Fibroblasts from these adhesions express the COX-2 enzyme while other fibroblasts in non-adhesion bearing areas do not express this enzyme (Saed and Diamond).

All tissues require angiogenesis for wound healing (Folkman, Braumald). Adhesions formation, a type of wound healing, is thus angiogenesis-dependent (Folkman Braumald 2000) (Wiczyk 1998)(Chegini 1997, Rout 2000). Recently, an experimental anti-angiogenic agent (TNP-470) was shown to inhibit adhesions in a murine model (Chiang 2001), suggesting that anti-endothelial agents may be efficacious in blunting the development of intra-abdominal adhesions. However, side-effects, such as absence of healing of abdominal wounds, precluded further investigations of this drug.

Several approaches have attempted to inhibit intra-abdominal adhesion formation, most with limited success (Montz 1994, Dizerega 1994, Rodgers 1997). Corticosteroids and anti-inflammatory agents have given inconsistent results (Panay 1999) while intra-abdominal crystalloids and dextran have not been shown to be effective (Soules 1982, Wiseman 1998). The most commonly used method of preventing post-operative adhesions uses a barrier agent or gel to separate damaged peritoneal surfaces for a minimum of 5-7 days. Hyaluronic acid and polyethylene glycol/polylactic acid barrier films have both been shown to reduce adhesion formation experimentally as well as clinically (Haney 1998, Wallwiener 1998, Rodgers 1998, Diamond 1998). Concerns about immune suppression, increased risk of infection with the intra-abdominal placement of these agents (Panay), and impaired healing of bowel anastomoses (Bowers et al 1999) remain.

These known approaches have been only moderately successful and more effective methods of prevention are necessary.

It has been known for some time that many of the common non-steroidal anti-inflammatory drugs (NSAIDs) modulate prostaglandin synthesis by inhibition of cyclooxygenases that catalyze the transformation of arachidonic acid—the first step in the prostaglandin synthesis pathway. However, the use of high doses of many common NSAIDs can produce severe side effects that limit their therapeutic potential. In an effort to reduce the unwanted side effects of common NSAIDS, it was discovered that two cyclooxygenases are involved in the transformation of arachidonic acid as the first step in the prostaglandin synthesis pathway. These enzymes have been termed cyclooxygenase-1 (COX-1) and cyclooxygenase-2 (COX-2)(Needleman, P. et al, J. Rheumatol., 24, SuppL49:6-8 (1997); Fu, J. Y., et al., J. Biol. Chem., 265(28):1673740 (1990)). COX-1 has been shown to beta constitutively produced enzyme that is involved in many of the non-inflammatory regulatory functions associated with prostaglandins. COX-2, on the other hand, is an inducible enzyme having significant involvement in the inflammatory process. Inflammation causes the induction of COX-2, leading to the release of prostanoids, which sensitize peripheral nociceptor terminals and produce localized pain hypersensitivity (Samad, T. A. et al, Nature, 410(6827):471-5 (2001)). Many of the common NSAIDs are now known to be inhibitors of both COX-1 and COX-2. Accordingly, when administered in sufficiently high levels, these NSAIDs affect not only the inflammatory consequences of COX-2 activity, but also the beneficial activities of COX-1. Recently, compounds that selectively inhibit COX-2 to a greater extent than the activity of COX-1 have been discovered. These new COX-2 inhibitors are believed to offer advantages that include the capacity to prevent or reduce inflammation while avoiding harmful side effects associated with the inhibition of COX-1, such as gastrointestinal and renal side effects, as well as bleeding from the inhibition of platelet aggregation.

The use of COX-2 inhibitors in the therapy of arthritis and related indications is known. U.S. Pat. No. 5,760,068 describes the use of COX-2 inhibitors for the treatment of rheumatoid arthritis and osteoarthritis. WO 00/32189 discloses the preparation of pharmaceutical compositions containing the COX-2 inhibitor celecoxib and the use of celecoxib for the treatment of rheumatoid arthritis or as a painkiller.

### SUMMARY OF THE INVENTION

The present invention provides a method for the minimization or prevention of adhesion formation during or following a surgical procedure. Such surgical procedures include, for example, orthopedic, plastic, abdominal, thoracic and cardiothoracic. The method comprises administering to the patient in need thereof a therapeutically effective amount of at least one COX-2 inhibitor, wherein the COX-2 inhibitor is not nimesulide. The COX-2 inhibitor can be administered before, during, or after surgery. In a preferred embodiment, the COX-2 inhibitor is celecoxib, also known as Celebrex (Pfizer).

In one embodiment the adhesion is between organ surfaces. As used herein, the term “organ surface” is intended to encompass any internal organ or tissue of a living animal including but not limited to the uterus, intestine, peritoneum, omentum, stomach, liver, kidneys, heart, and lung.

The present invention also provides a method of treating, preventing, or minimizing keloid formation, implant contractures including, but not limited to synthetic autologous, or heterologous implants such as used in breast, abdominal wall, cosmetic, orthopedic, hand, craniofacial or urologic implants, benign hypertrophic tumors, or scarring related to incision or cosmetic surgery. These methods comprise administering to a patient a therapeutically effective amount of at least one COX-2 inhibitor. The COX-2 inhibitor can be administered before, during, or after treatment.

The present invention further provides a medicament, and preparation of such a medicament, comprising a COX-2 inhibitor for use in treating, preventing or minimizing adhesions, keloids, scar formation and contractive formation.

As used herein the term “COX-2 inhibitor” refers to a non-steroidal drug that relatively inhibits the enzyme COX-2 in preference to COX-1. Preferred examples include celecoxib, parecoxib, rofecoxib, valdecoxib, meloxicam, and etoricoxib. In a preferred embodiment, the COX-2 inhibitor also possesses fibroblast inhibitory activity as measured by the methods set forth in Kusunoki et al., Arthritis & Rheumatism 46:3159-3167 (2002). In a most preferred embodiment, the COX-2 inhibitor further possesses antiangiogenic activity as measured, for example, by the methods set forth in Leahy et al., Cancer Research 62: 625-631 (2002), Masferrer et al., Cancer Research 60: 1306-1311 (2000), and Dicker et al., Am. J. Clin. Oncol. 24 (5): 438-442 (2001).

### BRIEF DESCRIPTION OF THE DRAWINGS

FIG. 1A is a photograph of adhesion formation in a control mouse at 10 days after laparotomy, cecal abrasions, and silicone patch placement. Note the extensive adhesion formation with attachment of cecum and omentum to the patch, preventing the patch to be visible (arrow).

FIG. 1B is a photograph of adhesion formation in a mouse treated with aspirin (1.2 mg/kg orally once a day) at 10 days after laparotomy, cecal abrasions, and silicone patch placement. There is dense adhesion formation over the silicone patch prohibiting the visualization of the patch (arrow).

FIG. 1C is a photograph of adhesion formation in a mouse treated with celecoxib (68 mg/kg orally twice a day) at 10 days after laparotomy, cecal abrasions, and silicone patch placement. There are fewer adhesions compared to both the control and non-selective COX inhibitors-treated groups. The entire patch is clearly visible without the presence of any peritoneal attachments (arrow).

FIG. 2 shows the effects of COX inhibitors on intra-abdominal adhesion formation. Aspirin, a non-selective COX-1 and COX-2 inhibitor, had no effect on intra-abdominal adhesion formation (p=N/S). Celecoxib and rofecoxib, selective COX-2 inhibitors, significantly decreased intra-abdominal adhesion formation by 74% and 91%, respectively (p<0.01). Celecoxib inhibition of adhesion formation was significantly greater than rofecoxib inhibition (p<0.05, star).

FIG. 3 shows CD31-immunostained sections of granulation tissue and muscle surrounding the silicone patch from representative mice of each group. Left portion of each image is the abdominal wall muscle, right portion is the, relevant, granulation tissue (see arrow in control image). The endothelium is stained red using a mAb specific for mouse CD31 (PECAM-1). There is clearly less endothelium present in the image of a celecoxib-treated animal as compared to that of the control animal. These findings were quantified and confirmed by statistical evaluation of the microvessel density obtained from these images (FIG. 4).

FIG. 4 shows microvessel density of granulation tissue surrounding the silicone patch. Celecoxib-treated animals had significantly lower microvessel density (3.8±0.8, star) than control animals (6.4±0.6, p<0.01), aspirin (13.3±1.0), naproxen (11.4±1.2), ibuprophen (9.9±1.1), indomethacin (11.3±1.5, all p<0.05), and rofecoxib (7.9±0.6, p<0.01). The microvessel density of all non-selective COX inhibitors was significantly higher than that of control animals (p<0.05).

FIG. 5 shows the effects of COX inhibitors on intra-abdominal adhesion formation.

### DETAILED DESCRIPTION OF THE INVENTION

We have now discovered methods of treating, preventing, and minimizing post-operative adhesion formation. The method comprises administering to a patient in need thereof a therapeutically effective amount of at least one COX-2 inhibitor, wherein the COX-2 inhibitor is not nimesulide.

The present invention can also be applied to the treatment, prevention, or minimization of keloid formation, implant contractures such as breast, benign hypertrophic tumors, and scarring in cosmetic or incision surgery by administering a COX-2 inhibitor.

The COX-2 inhibitor can be admitted to the patient before, during, or after surgery or at each of these times. In general, administration should be 12-48 hours prior to the time of surgery and for at least 24-48 hours post-surgery. Alternatively, the COX-2 inhibitor is administered 72 hours prior to surgery and continued at least 24 hours post-surgery. Preferably, at least 5 days post-surgery. In certain embodiments it might be desirable to administer the COX-2 inhibitor up to 2 weeks after surgery or even longer. When administered after abdominal surgery, the preferred method of administration is by suppository.

The COX-2 inhibitors of the present invention belong to the class of nonsteroidal anti-inflammatory drugs (NSAIDs). The term COX-2 inhibitor embraces compounds which selectively inhibit cyclooxygenase-2 over cyclooxygenase-1, and also includes pharmaceutically acceptable salts thereof. Also included within the scope of the present invention are compounds that act as prodrugs of cyclooxygenase selective inhibitors. As used herein in reference to COX-2 inhibitors, the term “prodrug” refers to a chemical compound that can be converted into an active COX-2 inhibitor by metabolic or simple chemical processes within the body of the subject.

COX-2 inhibitors that are useful in the invention can include compounds that are described in WO 02/102297.

Benzopyran COX-2 inhibitors useful in the practice of the present invention are described in U.S. Pat. Nos. 6,034,256 and 6,077,850.

In a preferred embodiment of the invention the COX-2 inhibitor is selected from the group of compounds, which includes celecoxib, valdecoxib, deracoxib, rofecoxib, etoricoxib, JTE-522, or a prodrug thereof.

Additional information about selected examples of the COX-2 inhibitors discussed above can be found as follows: celecoxib (CAS RN 169590 51 C-27791 SC-586531 and in U.S. Pat. No. 5,466,823); deracoxib (CAS RN 169590 4); rofecoxib (CAS RN. 162011 7); compound B-24 (U.S. Pat. No. 5,840,924); compound B-26 (WO 00/25779); and etoricoxib (CAS RN 202409 4, MK-663, SC-86218, and in TahIP 2).

Parecoxib (U.S. Pat. No. 5,932,598), which is a therapeutically effective prodrug of the tricyclic COX-2 inhibitor valdecoxib, (U.S. Pat. No. 5,633,272), may be advantageously employed as a source of a cyclooxygenase inhibitor. A preferred form of parecoxib is sodium parecoxib.

The compound ABT-963 that has been previously described in International Publication number WO 00/24719, is another tricyclic COX-2 inhibitor which may be advantageously employed.

The cyclooxygenase inhibitor can be selected from the class of phenylacetic acid derivative COX-2 inhibitors. A particularly preferred phenylacetic acid derivative COX-2 inhibitor that is described in WO 99/11605 is a compound that has the designation of COM 89 (CAS RN 346670 4). Compounds that have a similar structure are described in U.S. Pat. Nos. 6,310,099 and 6,291,523.

Further information on the applications of N-(2-cyclohexyloxynitrophenyl)methane sulfonamide (NS-398, GAS RN 123653 2), have been described by, for example, Yoshimi, N. et al., in Japanese J. Cancer Res., 90(4).R406-412 (1999); Falgueyret, J.-P. et al., in Science Spectra, available at: hftp://www.gbhap.com/Science,Spectra/20 article.htm (Jun. 6, 2001); and Iwata, K. et al., in Jpn. J. Pharmacol., 75(2):191-194 (1997).

Other materials that can serve as a COX-2 inhibitor include diarylmethylidenefuran derivatives that are described in U.S. Pat. No. 6,180,651.

Additional COX-2 inhibitors useful in the present invention, include N-(2cyclohexyloxynitrophenyl)methane sulfonamide, and (E) [(4methylphenyl)(tetrahydro oxo furanylidene) methyl]benzenesulfonamide.

Further COX-2 inhibitors that are useful in the present invention include darbufelone (Pfizer), CS-502 (Sankyo), LAS 34475 (Almirall Profesfarna), LAS 34555 (Almirall Profesfarma), S-33516 (Servier, see Current Drugs Headline News, at hftp://www.current-drugs.com/NEWS/Inflaml.htm, Oct. 4, 2001), 1 BMS-347070 (Bristol Myers Squibb, described in U.S. Pat. No. 6,180,651), MK-966 (Merck), L783003 (Merck), T-614 (Toyama), D-1 367 (Chiroscience), L-748731 (Merck), CT3 (Atlantic Pharmaceutical), CGP-28238 (Novartis), BF-389 (Biofor/Scherer), GR253035 (Glaxo Wellcome), 6-dioxo-9H-purin yi-cinnamic acid (Glaxo Wellcome), S-2474 (Shionogi); the compounds that are described in U.S. Pat. Nos. 6,310,079; 6,306,890 and 6,303,628 (bicycliccarbonyl indoles); U.S. Pat. No. 6,300,363 (indole compounds); U.S. Pat. Nos. 6,297,282 and 6,004,948 (substituted derivatives of benzosulphonamides); U.S. Pat. Nos. 6,239,173; 6,169,188, 6,133,292; 6,020,343; 6,071,954; 5,981,576 ((methylsulfonyl)phenyl furanones); U.S. Pat. No. 6,083,969 (diarylcycloalkano and cycloalkeno pyrazoles); U.S. Pat. No. 6,222,048 (diaryl (51-1)-furanones; U.S. Pat. No. 6,077,869 (aryl phenylhydrazines); U.S. Pat. Nos. 6,071,936 and 6,001,843 (substituted pyridines); U.S. Pat. No. 6,307,047 (pyridazinone compounds); U.S. Pat. No. 6,140,515 (3-aryl aryloxyfuran ones); U.S. Pat. Nos. 6,204,387 and 6,127,545 (diaryl pyridines); U.S. Pat. No. 6,057,319 (314diar@ hydroxy-2,5-dihydrofurans; U.S. Pat. No. 6,046,236 (carbocyclic sulfonamides); and U.S. Pat. Nos. 6,002,014; 5,994,381; and 5,945,539 (oxazole derivatives).

Preferred COX-2 inhibitors for the use according to the present invention include celecoxib (Celebrex™), rofecoxib (Vioxx™), meloxicam, piroxicam, deracoxib, parecoxib, valdecoxib (Bextra™), etoricoxib, a chromene derivative, a chroman derivative, N-(2cyclohexyloxynitrophenyl)methane sulfonamide, COX1 89, ABT963, JTE-522, pharmaceutically acceptable salts, prodrugs or mixtures thereof. The most preferred COX-2 inhibitors are celecoxib, parecoxib, valdecoxib, etoricoxib and rofecoxib. Valdecoxib is an alternative for patients with sulfa allergies.

According to a most preferred embodiment, celecoxib (Celebrex) or a pharmaceutically acceptable salt thereof is used. The term pharmaceutically acceptable salt includes salts that can be prepared according to known methods by those skilled in the art from the corresponding compound of the present invention, e.g. conventional metallic ion salts and organic salts.

The COX-2 inhibitor will generally be administered at the recommended dose of about 10-1000 mg/day, more preferably 50-400 mg/day for adults, and can be increased or decreased depending on clinical results. The administration can be carried out once or several times a day. The amount of COX-2 inhibitor can be adapted depending on age, body weight and/or possible other diseases of the patient.

Inert, pharmaceutically acceptable carriers used for preparing pharmaceutical compositions of the COX-2 inhibitors described herein can be either solid or liquid. Solid preparations include powders, tablets, dispersible granules, capsules, cachets and suppositories. The powders and tablets may comprise from about 5 to about 70% active ingredient. Suitable solid carriers are known in the art, e.g., magnesium carbonate, magnesium stearate, talc, sugar, and/or lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into conveniently sized molds, allowed to cool and thereby solidify.

Liquid preparations include solutions, suspensions and emulsions. As an example may be mentioned water or water-propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The COX-2 inhibitors may also be deliverable transdermally. The transdermal compositions can take the form of creams, lotions, aerosols and/or emulsions and can be included in a transdermal patch of the matrix or reservoir type as are conventional in the art for this purpose.

The suitability of a particular route of administration will depend on the pharmaceutical compositions (e.g., whether they can be administered orally without decomposing prior to entering the blood stream) and the disease being treated.

In a preferred form, the COX-2 inhibitor is administered in combination with a subcutaneously-implanted biodegradable, biocompatible polymeric implant or pump which releases the COX-2 inhibitor over a controlled period of time at a selected site. Examples of preferred polymeric materials include polyanhydrides, polyorthoesters, polyglycolic acid, polylactic acid, polyethylene vinyl acetate, and copolymers and blends thereof. See, Medical Applications of Controlled Release, Langer and Wise (eds.), 1974, CRC Pres., Boca Raton, Fla.; Controlled Drug Bioavailability, Drug Product Design and Performance, Smolen and Ball (eds.), 1984, Wiley, New York; Ranger and Peppas, 1983, J. Macromol. Sci. Rev. Macromol. Chem. 23:61; see also Levy et al., 1985, Science 228:190; During et al., 1989, Ann. Neurol. 25:351 Howard et al., 1989, J. Neurosurg. 71:105. In yet another embodiment, a controlled release system can be placed in proximity of the therapeutic target, i.e., the brain, thus requiring only a fraction of the systemic dose (see, e.g., Goodson, in Medical Applications of Controlled Release, 1989, supra, vol. 2, pp. 115-138).

Preferably the COX-2 inhibitor is administered by a variety of systemic and local methods including orally, intravenous and intracavity e.g., suppository. The COX-2 inhibitor may be administered by intracavity installation e.g., to the surface of the organs during the surgical procedure.

Preferably, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the COX-2 inhibitors will be regulated according to the judgment of the attending clinician (physician) considering such factors as age, condition and size of the patient as well as severity of the disease being treated.

The COX-2 inhibitor can be administered according to therapeutic protocols well known in the art. It will be apparent to those skilled in the art that the administration of the COX-2 inhibitor can be varied depending on the disease being treated and the known effects of the COX-2 inhibitor on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

### EXAMPLE

Methods

Adhesion Model

Seven to eight week-old male C57BL/6 mice (Jackson Laboratories, Bar Harbor, Me.) were housed five animals to a cage in a barrier room. Mice were acclimated to their environment for at least 72 hours prior to the initiation of each experiment and allowed food and water ad libitum. Animal protocols complied with the NIH Animal Research Advisory Committee guidelines and were approved by the Children's Hospital Institutional Animal Care and Use Committee. A standard adhesion model was performed as previously described (Chiang 2000). Specifically, a 5.0 mm×5.0 mm square piece of sterile silicone (Dow Corning, Midland, Mich.) was secured intra-abdominally to the right abdominal wall, lateral to the epigastric vessels, with two 7-0 proline sutures under isoflurane anesthesia. The cecum then was gently rubbed with two cotton swabs to promote adhesion formation.

Drug Treatment

After recovering from anesthesia, groups of mice were treated with either oral methylcellulose alone (100 μl, control) or an experimental drug mixed in 100 μl methylcellulose beginning on the day of surgery for 10 days. Groups of mice (n=4-16mice) treated with non-selective COX inhibitors received ibuprophen (30 mg/kg orally once a day) (Farag 1995), naproxen (10 mg/kg orally once a day) (Cicala 2000), aspirin (100 mg/kg orally once a day) (Jablonski 2002), or indomethacin (1 mg/kg orally once a day) (Yamamoto 2000). Mice treated with COX-2 selective inhibitors received either celecoxib (68 mg/kg orally twice a day) or rofecoxib (40 mg/kg orally once a day) (Laudanno 2001).

Quantification of Intra-Abdominal Adhesions

Ten and 35 days after surgery, mice were euthanized and adhesion formation over the silicone patch was measured by two blinded individuals based on a previously described scoring system (Chiang S, et al. J Ped Surg. 2000, 35, 189-196). The tenacity, type, and extent of adhesions to the patch, as well as the extent of adhesions to the cecum were graded on a scale of 0 to 4. The total adhesion score was the sum of the four individual scores (see table 1, below). A minimum score was 0 and the maximum score was 16. Tenacity was rated as none (0), adhesions fell apart (1), lysed with traction (2), lysed with blunt dissection (3), lysed with sharp dissection (4). Type was scored as none (0), filmy (1), mildly dense (2), moderately dense (3), very dense (4). Extent was measured as the percent of the patch covered by adhesion: 0% (0), <25% (1), 25-50% (2), 50-75% (3), >75% (4). Cecal adhesions were graded as none (0), adhesions fell apart (1), lysed with traction (2), lysed with blunt dissection (3), lysed with sharp dissection (4).

Table 1, below, demonstrates the scoring variables used to qualify and quantify the adhesions in control animals and animals treated with non-selective COX inhibitors and selective COX-2 inhibitors that have undergone a laparotomy, cecal abrasions, and had a silicone patch secured.

Immunohistochemistry

Silicone patches and abdominal wall were fixed at 4 degrees Celsius in 10% formalin overnight and washed with phosphate buffered saline. The specimens were then embedded in paraffin and 5 micron sections were cut. Endothelial cells were identified by antibody staining against the endothelial cell surface antigen CD31 (PECAM-1) and a diaminobenzidine chromogen (BD Biosciences Pharmingen, San Diego, Calif.). After deparaffinization and rehydration, sections were incubated with proteinase K (Roche, Basel, Switzerland) for 30 min at 37° C. Non-specific binding sites were blocked for 30 min at room temperature with TNB blocking buffer (NEN Life Sciences, Boston, Mass.). Primary CD31 antiserum (Pharmingen) was added to the sections overnight at 4° C. Slides were washed twice with phosphate buffered saline and secondary goat anti-mouse IgG antibody (Vector Laboratories, Burlingame, Calif.) was then added for 30 min at room temperature. After again washing twice with PBS the slides were incubated with streptavidin alkaline phosphatase solution (1:100) (Perkin-Elmer Life Sciences, Boston, Mass.) for 30 min at room temperature followed by biotinyl tyramide amplification dilutent (1:50) (Perkin-Elmer Life Sciences) for 10 min. After repeat incubation with alkaline phosphatase solution (1:100), for 30 min at room temperature, the chromagen, Vector Novar substrate kit (Vector Laboratories) was added. Slides were lightly counterstained with hematoxylin for imaging.

Imaging and Microvessel Density

Sections were taken from the abdominal wall, patch, and lesions at sacrifice on day 10. To quantitate vessel density, CD31 immunostained sections underwent digital image analysis using a Nikon Eclipse TE300 inverting photomicroscope with an attached Spot RT (Diagnostic Instruments Inc.) video camera linked to a personal computer. Images were imported directly to the image analysis program IP Lab (Scanalytics Inc., Fairfax, Va.). Sequential images were grabbed at 200× for the CD3.1 sections without overlapping. To calculate the volume fraction of stained regions, a color threshold was chosen for each image that distinguished between the stained areas and background. The proportion of the selected color was calculated as a percentage of total area and used as a measure of vessel density. Sections from 3 different mice for each of the study groups were examined and vascularization was scanned and quantified in 5 representative fields per section.

Statistical Analysis

Data are presented as mean±standard error of the mean. Statistical analysis of all results was performed by using the Student's two-tailed, unpaired t test for comparisons between groups (SigmaStat, SPSS, Chicago, Ill.). Differences were considered significant when p<0.05.

Results

All animals gained weight and appeared healthy. There were no signs of impaired wound healing or bleeding or gastro-intestinal complications.

Macroscopic Findings and Adhesion Score

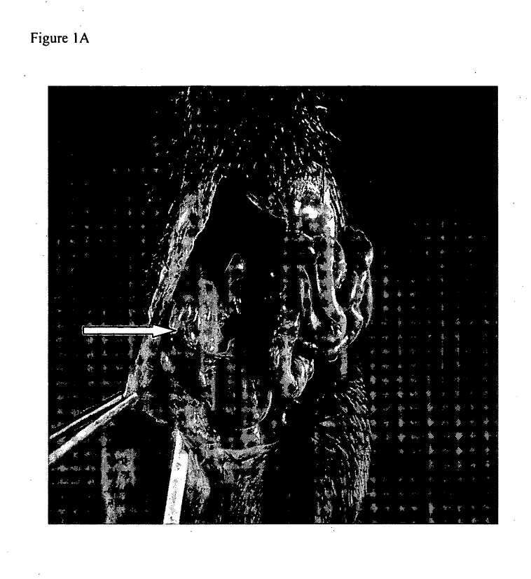

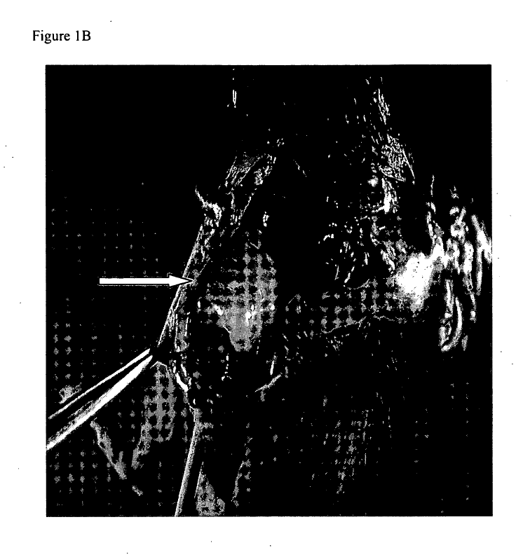

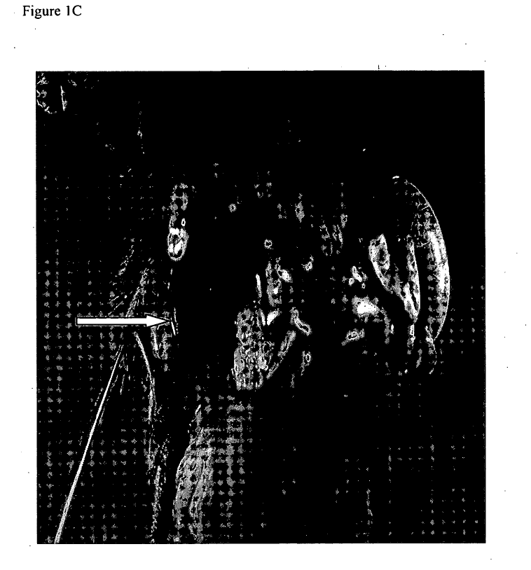

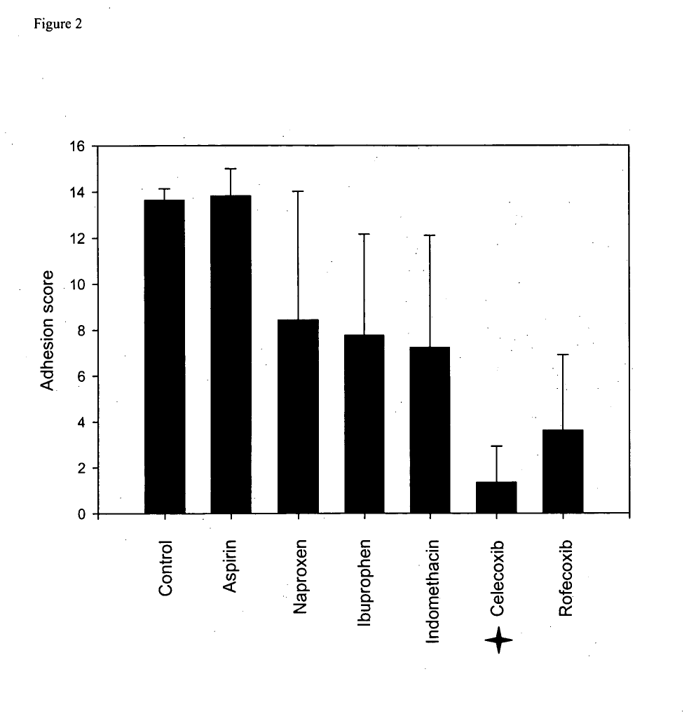

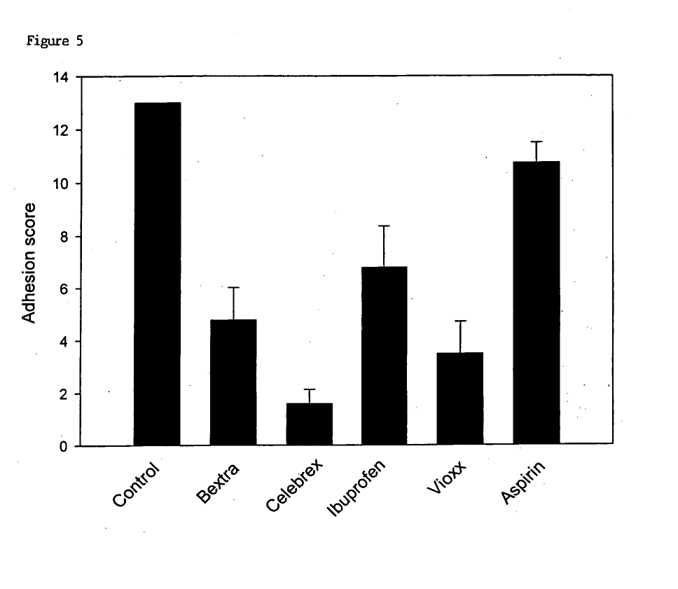

Macroscopic findings of representative animals in the control groups, the non-selective COX-inhibitors group (aspirin treated), and selective COX-2 inhibitors (celecoxib treated) are shown in FIG. 1. The intra-abdominal adhesion scores are demonstrated in FIGS. 2 and 5.

The intra-abdominal adhesion score in control mice was 13.6+/−0.49. The non-selective COX inhibitors, except for aspirin, significantly decreased intra-abdominal adhesions, compared to control animals (FIG. 2). Aspirin treated mice had a score of 13.8+/−0.4 (p=N/S), animals treated with naproxen, ibuprophen, and indomethacin had scores of 8.4+/−2.2, 7.8+/−1.4, and 7.3+/−1.7, respectively (p<0.01).

Selective COX-2 inhibitors significantly decreased intra-abdominal adhesion formation compared to both control animals as well as to animals treated with non-selective COX inhibitors (FIGS. 2 and 5). The adhesion score for rofecoxib treated animals was 3.6+/−1.1 (p<001 compared to control and p<0.05 compared to the non-selective COX inhibitors). Adhesions in the mice who received celocoxib were graded 1.36+/−0.5 (p<0.001 compared to control and non-selective COX inhibitors).

To determine whether the prevention of adhesions was durable, mice were treated for 10 days with celecoxib, rofecoxib, and aspirin and were observed for an additional 25 days before sacrifice. The adhesion scores for celecoxib, rofecoxib, and aspirin treated animals were 1.5+/−0.9 (p<0.01), 5.61-1.9 (p<0.01), and 12.2+/−1.2 (p=N/S) compared to control (13.4+/−1.0).

Immunohistochemistry and Microvessel Density

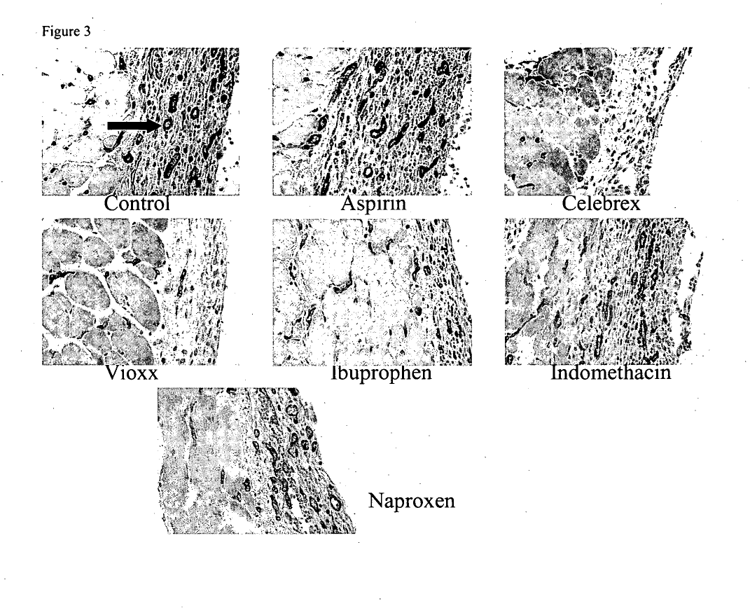

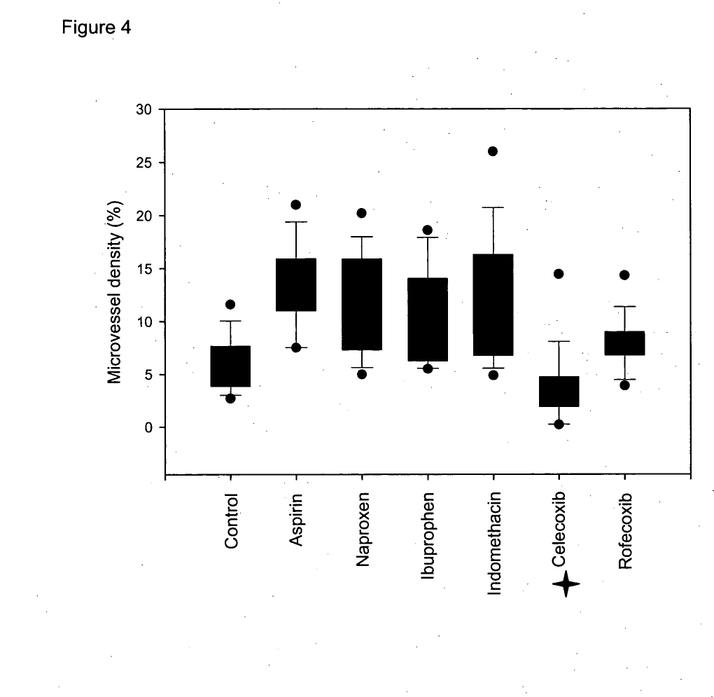

After determining the extent of intra-abdominal adhesions after laparotomy, cecal abrasion, and silicone patch placement in non-treated mice and mice treated with selective and non-selective COX inhibitors, the microvessel density of granulation tissue surrounding the patch were determined. These findings are depicted in FIGS. 3 and 4. Sections from representative mice stained with the endothelium-specific marker CD31 (FIG. 3) show clearly less endothelial cells (stained red) in the celecoxib-treated mice than the control animals.

Quantification of this data by calculating microvessel density and comparing control mice to mice treated with non-selective COX inhibitors of selective COX-2 inhibitors (FIG. 4) demonstrated that only the microvessel density in celecoxib-treated mice was lower than that of control mice (3.8±0.8 versus 6.4±0.6, p<0.01). The microvessel density for mice treated with the selective COX-2 antagonists celecoxib and rofecoxib (7.9±0.6) was significantly lower than the microvessel density in aspirin (13.3±1.0), naproxen (11.4±1.2), ibuprophen (9.9±1.1) and indomethacin (11.3±1.5) (p<0.05). Furthermore, comparing celecoxib and rofecoxib revealed significantly lower microvessel density scores in celecoxib treated animals (p<0.01). The microvessel density of all non-selective COX inhibitors was significantly higher than that of control animals (p<0.05).

All references cited below and described herein are incorporated herein by reference.

1. Ellis, H., The clinical significance of adhesions: focus on intestinal obstruction. Eur. J. Surg. 577: 5, 1997.

2. Ellis, H., Moran, B. j., Thompson, J. N., Parker M. C., Wilson, M. S., Menzies, D., McGuire, A., Lower, A. M., Hawthorn, R J, Obrien, F., Buchan, S., Crowe, A. Adhesion-related hospital readmissions after abdominal and pelvic surgery: a retrospective cohort study. Lancet 353: 1476, 1999.

3. Menzies, D., Ellis, H. Intestinal obstruction from adhesion—How big is the problem? Ann. R. Coll. Surg. Enl. 72: 60, 1990.

4. Menzies D, Parker M, Hoare R, Knight A. Small bowel obstruction due to postoperative adhesions: treatment patterns and associated costs in 110 hospital admissions. Ann R Coll Surg Engl. 83:40-6, 2001

5. Saed, G. M, Munkarah, A. R., Diamond, M. P. Cyclooxygenase-2 is expressed in human fibroblasts isolated from intraperitoneal adhesions but not from normal peritoneal tissues Fertil. Steril. 79:1404, 2003.

6. DiZerega, G. S. and Campeau, J. D. Peritoneal repair and post-surgical adhesion formation. Human Reproduction Update 7: 547, 2001

7. Ray, N. F., Denton, W. G., Thamer, M., et al. Abdominal adhesiolysis: Inpatient care and expenditures in the United States in 1994. J. Am. Coll. Surg. 186:1 1998.

8. Chegini, N. The role of growth factors in peritoneal healing: transforming growth factor beta (TGF-beta). Eur. J. Surg.; 577: 17, 1997.

9. Wiczyk, H. P., Grow, D. R., Adams, L. A., O'Shea, D. L., Reece, M. T. Pelvic adhesions contain sex steroid receptors and produce angiogenesis growth factors. Fertil. Steril. 69: 511, 1998.

10. Rout, U. K., Oommen, K., Diamond, M. P. Altered expressions of VEGF mRNA splice variants during progression of uterine-peritoneal adhesions in the rat. Am J Reprod Immunol. 43: 299, 2000.

11. Montz, F., Holschneider, C., Bozuk, M., et al. Interleukin 10: Ability to minimize postoperative intraperitoneal adhesion formation in a murine model. Fertil. Steril. 61: 1136, 1994.

12. DiZerega, G. S., Campeau, J., Use of instillates to prevent intraperitoneal adhesions; crystalloid and dextran. Infert. Reprod. Med. Clin. 5: 463, 1994.

13. Rodgers, K., Johns, D., Giris, W., et al. Reduction of adhesion formation with hyaluronic acid after peritoneal surgery in rabbits. Fertil. Steril. 67?, 553, 1997.

14. Diamond, M. P., Cunningham, C. B., Dizerega, G. S., et al. A model for sidewall adhesions in the rabbit: Reduction by an absorbable barrier. Microsurg. 8: 197, 1987.

15. Chiang, S. C, Cheng, C. H., Moulton, K. S., Kasqnica, J. M., Moulton, S. L. TNP-470 inhibits intrabdominal adhesions formation. J. Ped. Surg. 35: 189, 2000.

16. Guvenal, T., Cetin, A., Ozdemir, H., Yanar, O., Kaya, T. Prevention of postoperative adhesion formation in rat uterine horm model by nimesulide: a selective COX-2 inhibitor. Hum. Reprod. 16:1732, 2001.

17. Leahy, K. M., Koki, A. T., Masferrer, J. L. Role of cyclooxygenases in angiogenesis. Curr. Med. Chem. 7: 1163, 2000.

18. Masferrer, J. L., Koki, A., Seibert, K. COX-2 inhibitors. A new class of antiangiogenic agents. Ann. NY. Acad. Sci. 889: 84, 1999.

Masferrer, J. L., Leahy, K. M., Koki, A. T., Zweifel, B. S., Settle, S. L., Woemer, B. M., Edwards, D. A., Flickinger, A. G., Moore, R. J., Seibert, K. Antiangiogenic and antitumor activities or cyclooxygenase-2 inhibitors. Cancer Res. 60: 1306, 2000.

20. Dicker, A. P., Williams, T. L., Grant, D. S. Targeting angiogenic processes by combination rofecoxib and ionizing radiation. Am. J. Clin. Oncol. 24: 438, 2001.

21. Leahy, K. M., Omberg, R. L., Wang, Y., Zweifel, B. S., Koki, A. T., Masferrer, J. L. Cyclooxygenase-2 inhibition by celecoxib reduces proliferation and induceds apoptosis in angiogenic endothelial cells in vivo. Cancer. Res 62: 625, 2002.

22. Panay, N., Lower, A. M. New directions in the prevention of adhesion in laparoscopic surgery. Curr. Opin. Obstet. Gynecol. 11: 379, 1999.

23. Soules, M., Dennis, L., Bosarge, A., Moore, D. The prevention of postoperative pelvic adhesions: an animal study comparing barrier methods with dextran 70. Am. J. Obstet. Gynecol. 143: 829, 1982.

24. Wiseman, D. M., Trout, J. R., Diamond, M. P. The rates of adhesion development and the effects of crystalloid solution on adhesion development in pelvic surgery. Fertil. Steril. 70: 702, 1998.

25. Haney, A. F., Doty, E. A barrier composed of chemically cross-linked hyaluronic acid (Incert) reduces postoperative adhesion formation. Fertil. Steril. 70: 145, 1998.

26. Wallwiener, D., Meyer, A., Bastert, G. Adhesion formation of the parietal and visceral peritoneum: an explanation for the controversy on the use of autologous and alloplastic barriers? Fertil. Steril 68: 132, 1998.

27. Rodgers, K., Cohn, D., Hotovely, A., Pines, E., Diamond, M. P., dizerga, G. Evaluation of polyethylene glycol/polylactic acid films in the prevention of adhesions in the rabbit adhesion formation and reformation sidewall models. Fertil. Steril. 69: 403, 1998.

28. Diamond, M. P. Reduction of de novo postsurgical adhesions by intraoperative pre-coating with Sepracoat (HAL-C) solution: a prospective, randomized, blinded, placebo-controlled multicentre study. The Sepracoat Adhesion Study Group. Fertil. Steril. 69: 1067, 1998.

29. Rout, U. K., Oommen, K., Diamond, M. P. Altered expressions of VEGF mRNA splice variants during progression of uterine-peritoneal adhesions in the rat. AJRI 43: 299, 2000.

30. Wiczyk, H. P., Grow, D. R., Adams, L. A., O'Shea, D. L., Reece, M. T. Pelvic adhesions contain sex steroid receptors and produce angiogenesis growth factors. Fert. Steril. 69: 511, 1998.

31. Chegini, N. The role of growth factors in peritoneal healing: transforming growth factor beta (TGF-beta). Eur. J. Surg. 163: 17, 1997.

32. Farag, M. M., Salama, M. A., Abou-Basha, L. Experimental murine schistosomiasis: reduced hepatic morbidity after pre- and/or post-infection treatment with ibuprofen or diclofenac sodium. Ann. Trop. Med. Parasitol. 89: 497, 1995.

33. Cicala, C., Ianaro, A., Fiorucci, S., Calignano, A., Buci, M., Gerli, R., Santucci, L., Wallace, J. L., Cirino, G. NO-naproxen modulates inflammation, nociception and downregulates T cell response in rat Freund's adjuvant arthritis. Br. J. Pharmacol. 130: 1399, 2000.

34. Jablonski, P., Howden, B. O. Oral buprenorphine and aspirin analgesia in rats undergoing liver transplantation. Lab. Anim. 36: 134, 2002.

35. Laudanno, O. M., Cesolari, J. A., Esnarriaga, J., Rista, L., Piombo, G., Maglione, E., Aramberry, L., Sambrano, J., Godoy, A., Rocaspana, A. Gastrointestinal damage induced by celocoxib and rofecoxib in rats Dig. Dis. Sci. 46: 779, 2001.

36. Yamamoto, T., Sakashita, Y., Nozaki-Taguchi, N. Anti-allodynic effects or oral cox-2 selective inhibitor on post-operative pain in the rat. Can. J. Anaesth. 47: 354, 2000.

37. Bowers D, Raybon R B, Wheeless C R Jr. Hyaluronic acid-carboxymethylcellulose film and perianastomotic adhesions in previously irradiated rats. Am J Obstet Gynecol. 181:1335-7, 1999.

38. Wilkinson-Berka J L, Alousis N S, Kelly D J, Gilbert R E. COX-2 inhibition and retinal angiogenesis in a mouse model of retinopathy of prematurity. Invest Ophthalmol Vis Sci. 2003 March; 44(3):974-9.

39. Tsujii, M, Kawano, S, Tsuji, S, et al (1998) Cyclooxygenase regulates angiogenesis induced by colon cancer cells Cell 93,705-716

40. Ghosh, A K, Hirasawa, N, Niki, H, Ohuchi, K. (2000) Cyclooxygenase-2-mediated angiogenesis in carrageenin-induced granulation tissue in rats J Pharmacol Exp Ther 295,802-809

TABLE I

| Score | Tenacity | Type | Extent (%) | Cecal |
|---|---|---|---|---|
| 0 | None | None | 0 | None |
| 1 | Fell apart | Filmy | <25 | Fell apart |
| 2 | Lysed, taction | Mildly dense | 25-50 | Lysed, traction |
| 3 | Lysed, blunt dissection | Moderately | 50-75 | Lysed, blunt |
| | | dense | | dissection |
| 4 | Lysed, sharp | Very dense | >75 | Lysed, sharp |
| | dissection | | | dissection |

We claim:

1. A method of treating, preventing, or minimizing adhesion formation during or following a surgical procedure comprising administering to a patient in need thereof a therapeutically effective amount of at least one COX-2 inhibitor, wherein the COX-2 inhibitor is not nimesulide.

2. A method of treating, preventing, or minimizing keloid formation during or following a surgical procedure comprising administering to a patient in need thereof a therapeutically effective amount of at least one COX-2 inhibitor.

3. A method of treating or preventing benign hypertrophic tumors comprising administering to a patient in need thereof a therapeutically effective amount of at least one COX-2 inhibitor.

4. A method of treating, preventing, or minimizing scar formation during or following a surgical procedure comprising administering to a patient in need thereof a therapeutically effective amount of at least one COX-2 inhibitor.

5. A method of treating, preventing, or minimizing contracture formation during or following implantation of synthetic, autologous, or heterologous implants comprising administering to a patient in need thereof a therapeutically effective amount of at least one COX-2 inhibitor.

6. The method of claim 1 wherein the adhesion is between organ or tissue surfaces.

7. The method of claim 4 wherein the surgical procedure is cosmetic or by incision.

8. The method of claim 5 wherein the implant is a breast implant, implant used in cosmetic surgery, abdominal implant, urologic implant, or an orthopedic implant.

9. The method of claims 1-5 wherein the COX-2 inhibitor is selected from the group consisting of celecoxib, parecoxib, rofecoxib, valdecoxib, meloxicam, and etoricoxib.

10. The method of claims 1-5 wherein the COX-2 inhibitor is administered to said patient before surgery.

11. The method of claims 1-5 wherein the COX-2 inhibitor is administered by applying to organ surfaces during surgery.

12. The method of claims 1-5 wherein the COX-2 inhibitor is administered after surgery.

13. The method of claim 12 wherein the COX-2 inhibitor is administered by use of a suppository.

14. A method of treating, preventing, or minimizing adhesion formation during or following a surgical procedure comprising administering to a patient in need thereof a therapeutically effective amount of celecoxib.

15. A method of treating, preventing, or minimizing keloid formation during or following a surgical procedure comprising administering to a patient in need thereof a therapeutically effective amount of celecoxib.

16. A method of treating or preventing benign hypertrophic tumors comprising administering to a patient in need thereof a therapeutically effective amount of celecoxib.

17. A method of treating, preventing, or minimizing scar formation during or following a surgical procedure comprising administering to a patient in need thereof a therapeutically effective amount of celecoxib.

18. A method of treating, preventing, or minimizing contracture formation during or following implantation of synthetic, autologous, or heterologous materials comprising administering to a patient in need thereof a therapeutically effective amount of celecoxib.
